(19) United States
(12) Patent Application Publication
(10) Pub. No.: US 2022/0215353 A1
(43) Pub. Date: Jul. 7, 2022

(54) METHODS FOR ENHANCED FOOD SAFETY FOR FOOD STORAGE AND PREPARATION

(71) Applicant: Avery Dennison Retail Information Services LLC, Mentor, OH (US)

(72) Inventor: Jeanne DUCKETT, Franklin, OH (US)

(21) Appl. No.: 17/611,268

(22) PCT Filed: May 14, 2020
(86) PCT No.: PCT/US2020/032869

§ 371 Date: Nov. 15, 2021

Related U.S. Application Data
(60) Provisional application No. 62/849,351, filed on May 17, 2019.

(51) Int. Cl. G06Q 10/00; G06Q 10/08; G01N 33/02

(52) U.S. Cl. CPC G06Q 10/30; G01N 33/02; G06Q 10/0832

(57) ABSTRACT

Methods for food traceability are disclosed, which enhance food safety. In one embodiment, methods are disclosed for tracing a temperature profile of a food product from an origin to an ultimate disposition. In another embodiment, methods are disclosed for identifying a recall for a food item from an origin to an ultimate disposition are disclosed. In yet other embodiments, methods are disclosed for tracing a food item from a food service location to a consumer.

### CROSS-REFERENCE TO RELATED APPLICATIONS

This application claims the benefit of U.S. Application No. 62/849,351, filed May 17, 2019, the entire disclosure of which is incorporated herein by reference.

### FIELD

This invention relates generally to food safety management. More particularly, it relates to systems and methods for food safety management, particularly in retail and food service.

### BACKGROUND

Hazard Analysis Critical Control Point (“HACCP”) is a management system in which food safety is proactively addressed through the analysis and control of biological, chemical, physical and radiological hazards from raw material production, procurement and handling, to manufacturing, distribution and consumption of the finished product. HACCP attempts to avoid hazards in the chain of custody in the food supply chain rather than attempting to inspect finished products for the effects of those hazards. One of the focus areas for HACCP in food supply chain is retail and food service, including retail food markets with pre-packaged prepared foods, special order foods, and food delivered to the consumer by the retailer, restaurants, and crowd sourcing services (such as Uber Eats, GrubHub, DoorDash, Tapingo, Deliveroo, Caviar, and the like).

While it is recognized as a needed system, HACCP has proven burdensome for individuals and organizations in terms of implementation, recordkeeping, and verification. To facilitate the HACCP process and correct product handling issues, including recalled products, the HACCP has been integrated with Internet of Things (IoT) devices in the food processing area—including a Smart Prep Table, process map tools that describe food handling, improved receiving and shipping procedures, IoT temperature sensors, RFID readers, and smart printers. The HACCP system keeps a record of the process flow and the verification logs for the HACCP steps that were followed. In the US, for example, the system is capable of connecting to the cdc.gov feed for food recalls (voluntary and mandatory), including both food recalls by the FDA and USDA. When given a globally unique item identifier and business function, the system is also capable of generating a standard label with minimum input from the users.

Thus, there is a need for systems and methods to enable those in the food supply chain, especially those who handle and prepare the food products for consumers, to remove unsafe food products before they reach the consumer and/or alert consumers of potential issues with the safety of the food products. The invention is directed to these, as well as other, important needs.

### BRIEF SUMMARY

In a first embodiment, methods of tracing a temperature profile of a food product from an origin to an ultimate disposition, comprise:

receiving at least one food item having a first set of data linked thereto;
wherein the first set of data comprises the temperature profile of the food item from the origin; and
wherein the first set of data is encoded in a machine-readable label; comparing the temperature profile of the food item with an acceptable temperature range;

if the temperature profile is within the acceptable temperature range, transforming the at least one food item into a food product;

if the temperature profile is outside the acceptable temperature range, transforming the at least one food item into a food waste;

generating a second set of data linked to the food product or food waste;

wherein the second set of data is encoded in the machine-readable label; and

using the first and second sets of data to generate traceability information for the food product or the food waste.

In another embodiment, methods of identifying a recall for a food item between an origin to an ultimate disposition, comprise:

receiving at least one food item having a first set of data linked thereto;
wherein the first set of data comprises an identity of the food item; and
wherein the first set of data is encoded in a machine-readable label;

comparing the identity of the food item with a food recall list to generate a recall status;

if the identity of the food item is not on the food recall list, transforming the at least one food item into a food product;

if the identity of the food item is on the food recall list, transforming the at least one food item into a food waste;

generating a second set of data linked to the food product or food waste;
wherein the second set of data is encoded in the machine-readable label; and

using the first and second sets of data to generate traceability information for the food product or the food waste.

In yet other embodiments, methods for tracing a food item from a food service location to a consumer, comprise:

receiving the food item at the food service location;

assigning a set of critical information to the food item;
wherein the set of critical information comprises at least one of a temperature profile and a food recall status;

using the set of critical information to generate traceability information relative to the food item; and

encoding the traceability information in a machine-readable label.

In yet another embodiment, methods of ensuring employee compliance during preparation of a food product, comprise:

verifying the identity of an employee (employee ID, biometric scan, facial recognition) using an internet-of-thing printer;

evaluating compliance of the employee using the internet-of-thing printer;
wherein the compliance is selected from the criteria consisting of:
   training compliance;
   handwashing compliance;
   body temperature within a safe and healthy range; and
   combinations thereof;
wherein the handwashing compliance further comprising using a smart device; and

if the employee is compliant, permitting the employee to begin preparation of the food product.

As will be realized, the subject matter described herein is capable of other and different embodiments and its several details are capable of modifications in various respects, all without departing from the claimed subject matter. Accordingly, the drawings and description are to be regarded as illustrative and not restrictive.

### BRIEF DESCRIPTION OF THE DRAWINGS

FIG. 1 shows an example of preconfigured data in accordance with the disclosed architecture.

FIG. 2 shows a schematic view of one possible embodiment of a foodservice kitchen with locations uniquely identified in accordance with the disclosed architecture.

FIG. 3A shows a flowchart of one method for receiving the food product item in accordance with the disclosed architecture relating to temperature compliance.

FIG. 3B shows a flowchart of one method for receiving the food product item in accordance with the disclosed architecture relating to a food recall.

FIG. 4 shows an example of an approved vendor list in accordance with the disclosed architecture of FIG. 3A.

FIG. 5 shows an example of an as-received label for a food item in accordance with the disclosed architecture.

FIG. 6 shows a perspective view of a sample user interface for the receive process in accordance with the disclosed architecture.

FIG. 7 shows a flowchart of one example of a method of processing a food product item in accordance with the disclosed architecture.

FIG. 8A shows a flowchart of the initial steps of one example of a method of transformation of the food product item in accordance with the disclosed architecture.

FIG. 8B shows a flowchart of the remaining steps of the method of transformation of the food product item of FIG. 8A in accordance with the disclosed architecture.

FIG. 9 shows one example of preconfigured prep item table data in accordance with the disclosed architecture.

FIG. 10 shows a perspective view of one example of a sample input screen in accordance with the disclosed architecture.

FIG. 11 shows a perspective view of event data generated for transformation in accordance with the disclosed architecture.

FIG. 12 shows a front perspective view of one example of a label for an intermediate food product item in accordance with the disclosed architecture.

FIG. 13 shows a front perspective view of one example of a shipping label for a consumer food product item in accordance with the disclosed architecture.

FIG. 14 shows a front perspective view of one example of a card with traceability information that may be served with a food product item in a restaurant or other food service setting in accordance with the disclosed architecture.

FIG. 15 shows a front perspective view of one example of a quick serve label with traceability information that may be served with a food product item in accordance with the disclosed architecture.

FIG. 16 shows a front perspective view of a web page for consumer food product information in accordance with the disclosed architecture.

FIG. 17 shows a front perspective view of an RFID device in accordance with the disclosed architecture.

FIG. 18 shows a front perspective view of a food freshness printer in accordance with the disclosed architecture.

FIG. 19 shows the tracking of different possible locations for the food product in accordance with the disclosed architecture.

FIG. 20 shows an HACCP food process map in accordance with the disclosed architecture.

FIG. 21 shows an exemplary label prepared for an end consumer consumption in accordance with the disclosed architecture.

FIG. 22 shows an exemplary label for an expired food item in accordance with the disclosed architecture.

FIG. 23 shows an exemplary label for a food item subject to a recall in accordance with the disclosed architecture.

FIG. 24 shows an exemplary web page containing information about a food product in accordance with the disclosed architecture.

FIG. 25 shows a process map with steps prior to food preparation in accordance with the disclosed architecture.

### DETAILED DESCRIPTION OF THE EMBODIMENTS

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof.

The following definitions are used herein to further define and describe the disclosure:

As used herein, the terms “comprises,” “comprising,” “includes,” “including,” “has,” “having” or any other variation thereof, are intended to cover a non-exclusive inclusion. For example, a process, method, article/composition, or apparatus that comprises a list of elements is not necessarily limited to only those elements but may include other elements not expressly listed or inherent to such process, method, article/composition, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

As used herein, the terms “a” and “an” include the concepts of “at least one” and “one or more than one.”

As used herein, the term “food item” refers to a food substance before processing (such as, for example, through mechanical, chemical, thermal, radiation, or the like means) is conducted on such food substance. Such processing may include, but is not limited to, thawing, heating, cooking (grilling, frying, baking, sautéing, searing, roasting, broiling, and the like), cooling, freezing, mixing with another food substance (blending, beating, combining, and the like), whipping, coating, cutting, chopping, dividing, assembling, irradiating, and the like and combinations (in any order) thereof.

As used herein, the term “food product” refers to a food substance after processing (such as, for example, through mechanical, chemical, thermal, radiation, or the like means) is conducted on such food substance. Such processing may include, but is not limited to, thawing, heating, cooking (grilling, frying, baking, sautéing, searing, roasting, broiling, and the like), cooling, freezing, mixing with another food substance (blending, beating, combining, and the like), whipping, coating, cutting, chopping, dividing, assembling, irradiating, and the like and combinations (in any order) thereof. It should be noted that a food substance may be a “food item” and a “food substance” depending upon whether or not it is the input into the process step (which would be referred to as the “food item”) or the output from the process step (which would be referred to as the “food product”). In certain processes, a food substance that is processed from an incoming food item but is not yet in its final state as food product will be referred to herein as an “intermediate food product” to indicate that further processing is expected on the food substance prior to its final disposition, such as for serving, sale, donation, or even waste.

As used herein, the term “food waste” refers to a food substance that is no longer wanted or needed by the user (such as, for example, by a food preparer, food service worker, distributor, retailer, or consumer) and includes, but is not limited to, recalled food substances and food that has experienced environmental or handling conditions that may render it unsafe and/or unhealthy for human or animal consumption.

As used herein, the term “recall,” in reference to a food substance, refers to an action or actions by a food supplier, transporter, processor, distributor, retailer (including, for example, stores, food pantries, restaurants, and food delivery services), or users (for example, consumers of the food substance and food service workers in institutions, such as educational facilities, food pantries, healthcare facilities, veterinary facilities, animal handling facilities, and government facilities) taken to remove from sale, distribution, and/or consumption of foods that may pose a safety and/or health risk to human or animal that consumes the food substance. The safety and/or health risk may include an expired food substance, a food substance that experienced a temperature range outside a safe range (possibly for a certain period of time), or a recalled food substance (such as, for example, contamination by allergens (including eggs, nuts, peanuts, and the like), microbes (including bacterial, viral, fungal, and the like), foreign substances (toxic chemicals, glass, plastic, hair, feces, and the like), and the like.

As used herein, the term “temperature profile,” in reference to a food substance, refers to the range of temperature from the lowest temperature to the highest temperature experienced by a food substance, usually from its origin to the point of reading the range. The temperature profile may also include the duration that the food substance experienced a particular temperature or temperatures, which may be useful for determining the safety (such as, for example, an elevated temperature that leads to early spoilage) and/or quality (such as, for example, an episode of freezing that leads to inferior quality) of the food substance.

As used herein, the term “chain of custody,” in reference to a food substance in the context of supply chains, is a certification mechanism that enables food substances to come with a digital passport that serves as a verifiable transcript of the food substance's life-cycle and journey. The chain of custody chronologically documents (physically or electronically) the ingredients, components and final products, their condition from production to destination as well as along the distribution lines recording any disruption in the product's environment that could negatively influence the products quality or performance. This documentation preferably should be precise enough to also be validated by a court.

Accordingly, in a first embodiment, methods of tracing a temperature profile of a food product from an origin to an ultimate disposition, comprise:

receiving at least one food item having a first set of data linked thereto;
wherein the first set of data comprises the temperature profile of the food item from the origin; and
wherein the first set of data is encoded in a machine-readable label; comparing the temperature profile of the food item with an acceptable temperature range;

if the temperature profile is within the acceptable temperature range, transforming the at least one food item into a food product;

if the temperature profile is outside the acceptable temperature range, transforming the at least one food item into a food waste;

generating a second set of data linked to the food product or food waste;

wherein the second set of data is encoded in the machine-readable label; and

using the first and second sets of data to generate traceability information for the food product or the food waste.

In another embodiment, methods of identifying a recall for a food item between an origin to an ultimate disposition, comprise:

receiving at least one food item having a first set of data linked thereto;
wherein the first set of data comprises an identity of the food item; and
wherein the first set of data is encoded in a machine-readable label; comparing the identity of the food item with a food recall list to generate a recall status;

if the identity of the food item is not on the food recall list, transforming the at least one food item into a food product;

if the identity of the food item is on the food recall list, transforming the at least one food item into a food waste;

generating a second set of data linked to the food product or food waste;
wherein the second set of data is encoded in the machine-readable label; and

using the first and second sets of data to generate traceability information for the food product or the food waste.

In yet other embodiments, methods for tracing a food item from a food service location to a consumer, comprise:

receiving the food item at the food service location;

assigning a set of critical information to the food item;
wherein the set of critical information comprises at least one of a temperature profile and a food recall status;

using the set of critical information to generate traceability information relative to the food item; and

encoding the traceability information in a machine-readable label.

In yet another embodiment, methods of ensuring employee compliance during preparation of a food product, comprise:

verifying the identity of an employee (employee ID, biometric scan, facial recognition) using an internet-of-thing printer;

evaluating compliance of the employee using the internet-of-thing printer;
wherein the compliance is selected from the criteria consisting of:
   training compliance;
   handwashing compliance;
   body temperature within a safe and healthy range; and
   combinations thereof;
wherein the handwashing compliance further comprising using a smart device; and

if the employee is compliant, permitting the employee to begin preparation of the food product.

In certain embodiments, the methods further comprise the step of providing the traceability information for the food product or food waste to a user.

In certain embodiments, the ultimate disposition of the food product is consumption by a consumer or a re-purposing (such as, for example, as a donation to a food pantry or food donation location). In alternative embodiments, the ultimate disposition of the food waste is a disposal (for example, because the food item was outside the acceptable temperature range or was subject to a product recall) or a return to a supplier (for example, because it is incorrect food item, an unacceptable food item from a quality perspective, or a recalled food item).

In certain embodiments, the first set of data further comprises a shelf life for each of the at least one food item and the second set of data further comprises an expiration date for the food product. The shelf life for each of the at least one food item may be used to calculate the expiration date for the food product.

In certain embodiments, the methods further comprise using the temperature profile to generate an alert about the food product to a user. In other embodiments, the methods further comprise using the recall status to generate an alert about the food product to a user. In yet other embodiments, the methods further comprise using the temperature profile, the recall status or a combination thereof to generate an alert about the food item for the consumer.

In certain embodiments, traceability information is at least one option selected from the group consisting of (a) a list of ingredients of the food product; (b) a source of the list of ingredients; (c) an identifying number for the food product; and (d) an expiration date for the food product.

In certain embodiments, the machine-readable label comprises at least one identifier selected from the group consisting a barcode, a radio-frequency identification (RFID) tag (also referred to as a label or inlay), and a quick response (QR) code.

In certain embodiments, the methods further comprise providing the recall status and traceability information for the food product or food waste to a user. In certain embodiments, the methods further comprise using the recall status to generate an alert about the food product to a user.

In certain embodiments, the set of critical information further comprises one or more for the food item: (a) a vendor identifier; (b) an identification number; (c) a description; (d) a storage location; (e) a shelf life; and (f) a weight.

In certain embodiments, the traceability information further comprises one or more of the following relative to the food item: (a) a list of ingredients; (b) a source of each of the list of ingredients; (c) an identifying number; and (d) an expiration date.

In certain embodiments, the methods further comprise assigning an initial destination to the food item and comprising the initial destination in the traceability information. The initial destination may be one of the consumer, a storage location, or a shipping location.

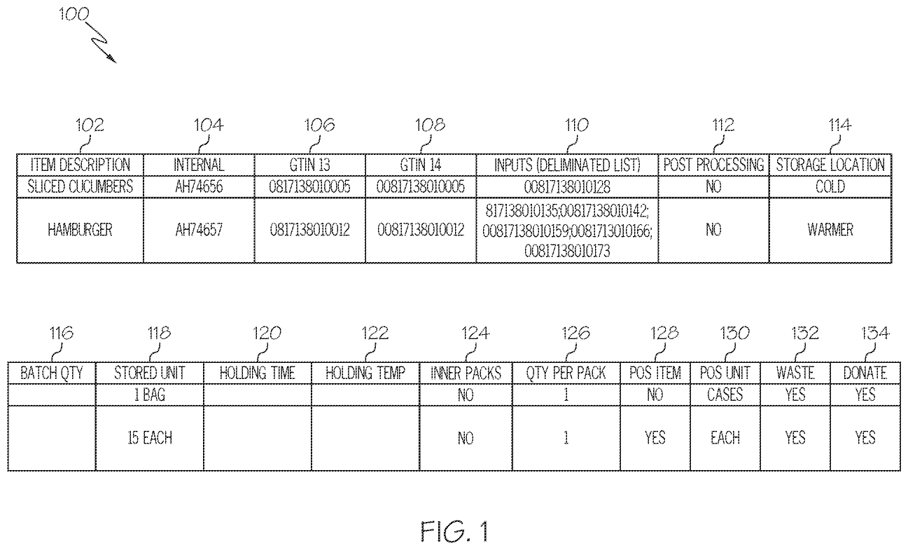

For representative examples, reference is now made to the Figures. Critical information can be any information deemed necessary for food traceability, such as the various types of information 100 disclosed in FIG. 1. For example, FIG. 1 shows critical information 100 such as an item description 102, internal tracking number 104, Global Trade Item Numbers (GTN13 and GTN14) 106 and 108, inputs (deliminated list) 110, post-processing requirements 112, storage location 114, batch quantity 116, stored unit 118, holding time 120, holding temperature 122, inner packs 124, quantity per pack 126, point-of-sale (POS) item 128, POS unit 130, eligibility for waste 132, eligibility for donation 134. In FIG. 1, for enhanced food safety, a more complete data set is given—including whether or not the food item or product has been received, whether or not there may be substitutions, and whether or not there may be donations permitted for both received food item and prepared food items. The table in FIG. 1 refers to both intermediate food products (such as the sliced cucumbers) and prepared food products such it only has one input item (such as the hamburger). It will be appreciated by one of ordinary skill in the art that the method of the present invention is not limited to the forgoing examples of critical information 100, and that other types of information, critical and non-critical, can also be included to suit user demand and/or preference.

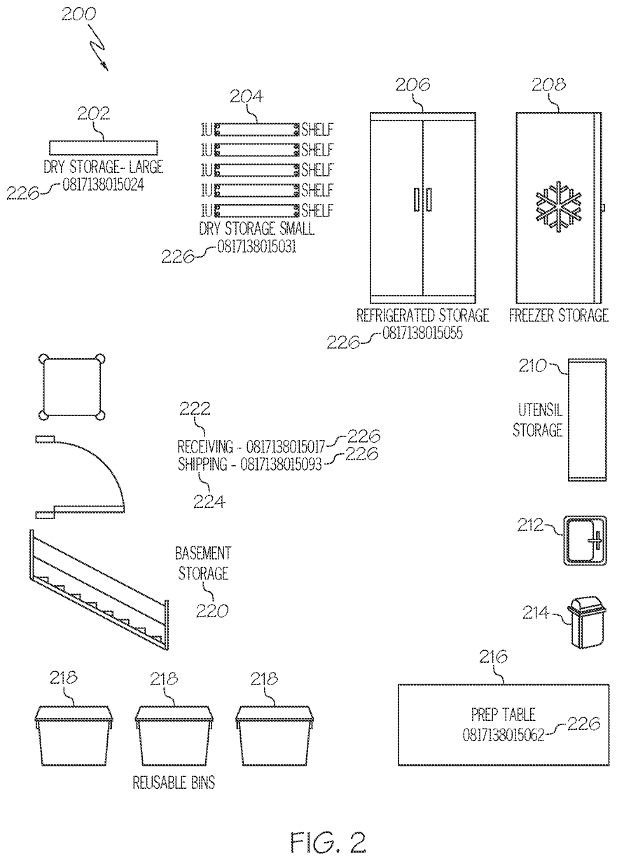

FIG. 2 is a schematic view of one potential embodiment of a foodservice kitchen 200 with locations, such as those typically associated with food preparation and/or storage, uniquely identified. For example, FIG. 2 discloses a relatively large area for dry storage 202, a small area for dry storage 204, a refrigerated storage 206, a freezer storage 208, a utensil storage 210, a sink 212, a trash receptacle 214, a preparation table 216, a plurality of reusable bins 218, a basement storage area 220, a receiving area 222, and a shipping area 224. Each of the various areas in the kitchen 200 are identified with a unique number 226, which provides a user with complete visibility into the food product transformation trail, as described more fully below.

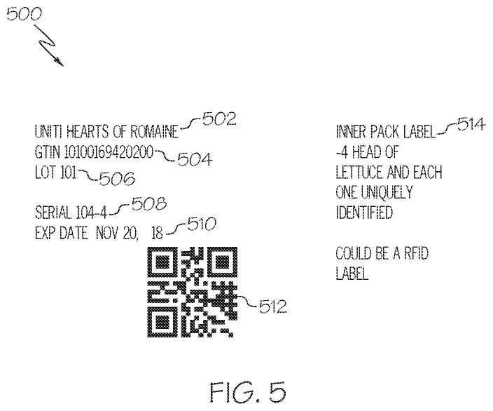

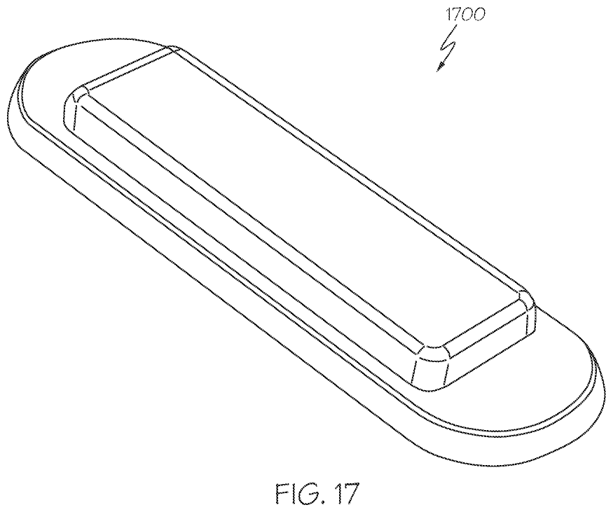

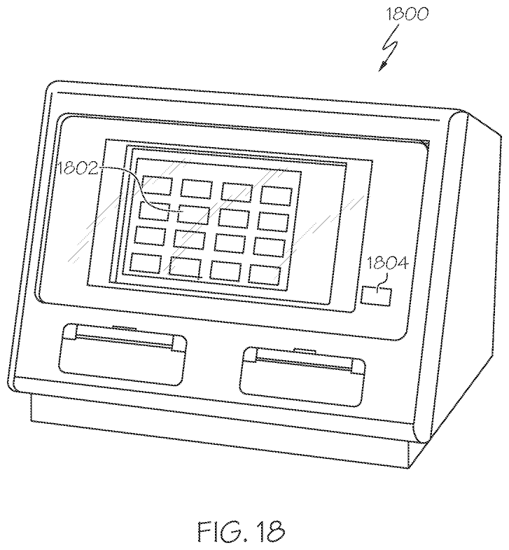

To initiate the process of food traceability, the user will receive incoming food products that are labelled, marked or otherwise tagged by an intelligent barcode printer 1800, such as the one depicted in FIG. 18. Printer 1800 is preferably equipped with a user display 1802, a barcode or QR code 1804 and/or an RFID reader 1700, such as the reader depicted in FIG. 17, or a smart device that is communicating with a barcode/RFID printer 1800, or any other suitable device as is known in the art, thereby creating a link to the chain of custody. More specifically, the incoming food products are preferably marked with a label 500, such as the label shown in FIG. 5, which includes a food product or item name 502, a unique identifier or GTIN 504, a batch/lot number 506, a serial number 508, a product freshness date 510, a QR code 512, an item description 514, and other information that is deemed necessary or useful by a user. By way of example, the product freshness date 510 could be one of many dates indicating product freshness and/or useful life including, without limitation, a pack date, an expiration date, a “use by” date and/or a “best by” date. The label 500 may also be a RFID label, or any other suitable label or code that could be scanned in and recorded and/or linked to a webpage or database.

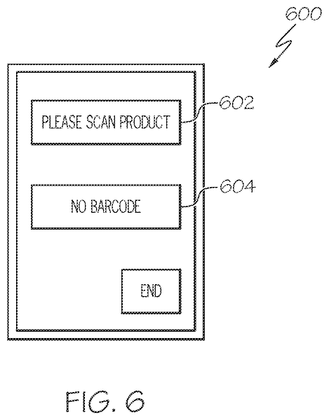

An example of one embodiment of a user interface 600 is shown in FIG. 6, wherein the user is prompted to scan a food product item at 602. Scanning the food product item at 602 generates the traceability data in the background as a byproduct of normal printer functions but is less intrusive to the user. On the other hand, if the food product item is not already marked with a barcode 604, or other code such as a QR code, RFID tag, etc., the user will have the option to scan a code for the respective food product item from a scan book (not shown) to generate and print a receiving label for the food product item, or to input the necessary information to generate and print said label.

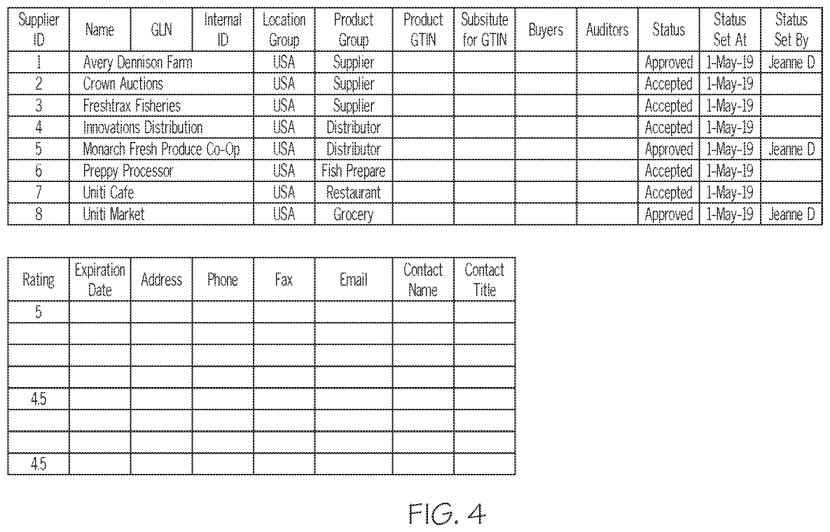

FIG. 4 describes the approved vendor listing the products that are received from each vendor including the approved substitution list for each product. Also holding times and temperature ranges for proper food safety are provided in the configuration data.

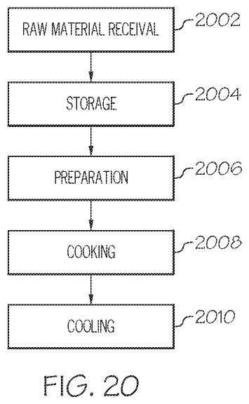

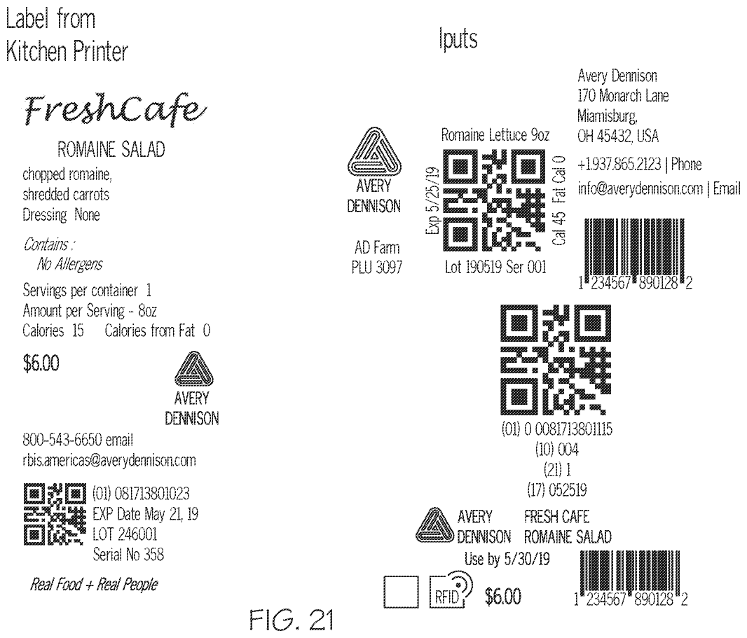

In FIG. 20 a HACCP food process map is shown. The first step 2002 is receiving material in compliance. The enhanced printer receive process ensures compliance with requirements, such as those set forth in the preconfigured data of FIG. 1 and approved vendor list of FIG. 4. During the normal course of printing labels for incoming food items, the printer verifies compliance and logs the data to a local or cloud server. The next step 2004 is the proper storage of a food item. In FIG. 1 the correct storage parameters are configured. When the user prints the receipt label, either the compliance is verified and logged or the non-compliant action taken by the user is logged. Step 2006 is the preparation of a food product verified with the printer. When the preparation label is printed, the cooking 2008, hot holding (not shown), and/or cooling 2010 actions are verified and logged, creating a link to the chain of custody. From this point, the after preparation action is logged for the food item, if an intermediate process food product returns to storage or the final food product is processed for sale, donated, or waste.

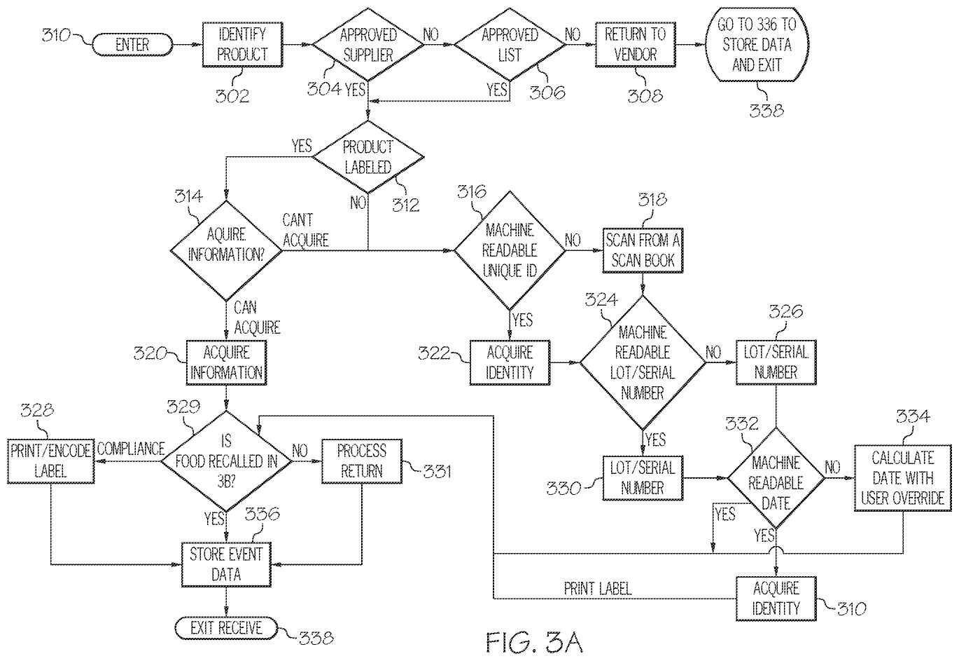

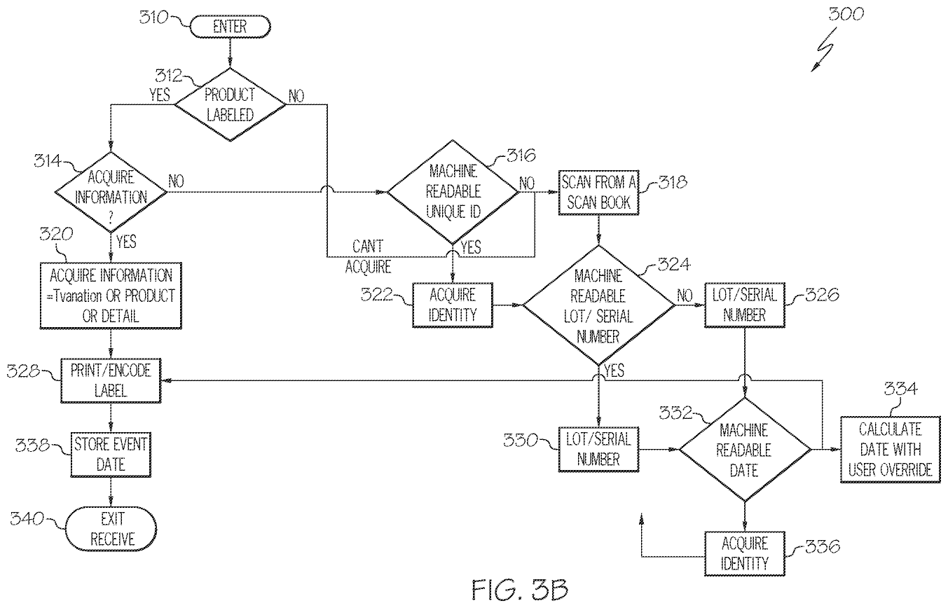

One possible example of the receive process 300 for food product items is generally depicted in FIG. 3A. FIG. 3A shows a flowchart of the method of tracing a temperature profile of a food product from its origin to an ultimate disposition that is initiated at 310 and then the product is identified as a product to be received in step 302. In 312 the product is checked for machine-readable information. If the product is labeled, the product information, which is stored in a barcode, RFID tag, or other machine-readable label, is read in 314. If the data is successfully read, the data is stored in 320 and the process continues to 306 where the item is verified that the vendor is approved to ship this item. If the item is a substitute the information for the alternate product is verified. If the product is not in compliance a return is processed and the data is logged and the process exits. In the case of a key drop receive or the case where the receiving location is unattended, this process proceeds without any user interaction. If the vendor and product is within compliance the process moves to 329 where the temperature is checked from the product data to be compliant. If the temperature is within compliance, the process then prints/encodes the received label 328 and stores the event data in the background 336 and the process exits 338. If the three critical elements: unique identification, product lot/serial number and date are not present in machine readable form (either barcode or RFID) the process determines if each individual element can be machine read or if not an alternate data entry process is used. One such alternate method is using a vision system on a smart device to read the product date and lot number. In 316 the user looks for a machine-readable unique identifier, if one is present the process continues to 336 to acquire the identity and moves to 306 to verify the vendor/product compliance and temperature compliance processing as above. If there is not a machine readable unique identity, then an identity is scanned from a prepared scan book in 318. Operation 318 is also the entry point from 314 in case of an acquisition error. The process then continues onto decision 324 where it is determined if there is a readable product batch or serial number. If yes, the machine read occurs in 330; if not the product is assigned a unique serial number from a combination of the device id and incrementing number. Next in 332 the user determines if there is a machine readable data. If there is one, it is read in 336; if there is no machine-readable data, the user is prompted with the current date plus the product life from the chart in FIG. 1. The user may override this date if required. The product identity is acquired in 336.

Another possible example of the receive process 340 for food product items is generally depicted in FIG. 3B. FIG. 3B shows a flowchart of the identifying a recall for a food item between an origin to an ultimate disposition that is initiated at 350 and then the product is identified as a product to be received in step 352. In 352 the product is checked for machine-readable information. If the product is labeled, the product information, which is stored in a barcode, RFID tag, or other machine-readable label, is read in 354. If the data is successfully read, the data is stored in 370 and the process continues to 346 where the item is verified that the vendor is approved to ship this item. If the item is a substitute the information for the alternate product is verified. If the product is not in compliance a return is processed and the data is logged and the process exits. In the case of a key drop receive or the case where the receiving location is unattended, this process proceeds without any user interaction. If the vendor and product is within compliance the process moves to 369 where the temperature is checked from the product data to be compliant. If the temperature is within compliance, the process then prints/encodes the received label 368 and stores the event data in the background 336 and the process exits 378. If the three critical elements: unique identification, product lot/serial number and date are not present in machine readable form (either barcode or RFID) the process determines if each individual element can be machine read or if not an alternate data entry process is used. One such alternate method is using a vision system on a smart device to read the product date and lot number. In 356 the user looks for a machine-readable unique identifier, if one is present the process continues to 376 to acquire the identity and moves to 346 to verify the vendor/product compliance and temperature compliance processing as above. If there is not a machine readable unique identity, then an identity is scanned from a prepared scan book in 358. Operation 358 is also the entry point from 354 in case of an acquisition error. The process then continues onto decision 364 where it is determined if there is a readable product batch or serial number. If yes, the machine read occurs in 370; if not the product is assigned a unique serial number from a combination of the device id and incrementing number. Next in 372 the user determines if there is a machine readable data. If there is one, it is read in 376; if there is no machine-readable data, the user is prompted with the current date plus the product life from the chart in FIG. 1. The user may override this date if required. The product identity is acquired in 376.

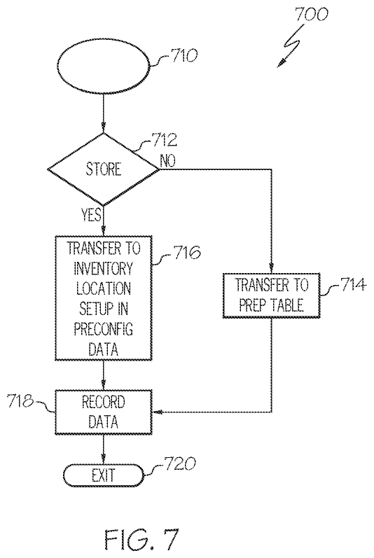

The next sub-process in the food service traceable process is the “put away” process. Generally stated, the put away process is designed to be relatively simple for the user while providing the necessary verification of the HACCP Food Flow Process. In the initial configuration, the storage location for each unique food product item is identified. After the food product item is received by a user, the user is prompted as to whether to store the food product item or not. If yes, then the food product item is stored in the designated area and the traceability information is generated in the background. If the answer is no, then the food product item is transferred to the prep table with the unique item information generated in the background. Specifically, the food product “put away” process 700 is illustrated at FIG. 7. The put away process initiates at 710 and proceeds to the decision at 712 as to whether to store the food product item or move the food product item directly to the prep table. If the food product item is to be stored, the necessary data for storage of the food product item is generated at 716. If, on the other hand, the food product item is not going to be stored, then the same is transferred to the prep table at 714. The two paths then converge at 718 where the necessary data is recorded and the process exits at 720. The inventory process highlights one of the advantages of using RFID technology in the process. For example, with RFID labeled inner packs and RFID readers mounted in the dry storage, the refrigerated storage, and the freezer storage, an up to date inventory with lot/serial numbers and relevant date information can be easily obtained and maintained.

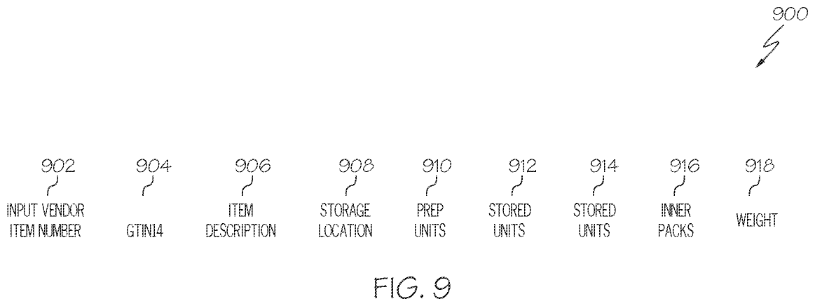

The next sub-process in the food service traceable process is the transformation process, regardless of whether the transformation process produces an end food product for consumption or an intermediate product that will be used at a later time to produce an end food product for consumption. It is important to keep the following key data elements of the food product in place during the transformation process: the unique identity, the batch/serial number, and date. In the transformation process, an intelligent food process printer 1800, such as the one depicted in FIG. 18, is used to select the food product item to be prepped. The prepped food items are preconfigured by the user with the table 900 shown in FIG. 9 mapping the received food products into prepped food items. Similar to the received product list depicted in FIG. 1, the prepped food items preferably have a unique identifier and a “use by” date. The use by date should be no later in time than the last use by date from the prepped food items. As best depicted in FIG. 9, the prepped food product items can further comprise an input vendor/item number 902, a GTIN14number 904, an item description 906, a storage location 908, a prep unit 910, a plurality of stored units 912, a shelf life 914, a plurality of inner packs 916, and a weight 918.

The process from 334 and 332 in FIG. 3A (or 374 and 372 in FIG. 3B) converges at 306 (or 346 in FIG. 3B) where the item is verified that the vendor is approved to ship this item. If the item is a substitute the information for the alternate product is verified. If the product is not in compliance a return is processed and the data is logged and the process exits. This action can happen in an unattended manner, capturing the correct chain of custody information and processing the return in an unmanned operation. If the vendor and product is within compliance the process moves to 329 in FIG. 3A (or 369 in FIG. 3B) where the temperature is checked from the product data to be compliant. If the temperature is within compliance, the process then prints/encodes the received label 328 in FIG. 3A (or 368 in FIG. 3B) stores the event data in the background and the process exits. From the table in FIG. 1, the printer prints/encodes the appropriate number of inner pack labels with an example label shown in FIG. 5. The printer can serialize the inter pack labels. The final step in the receive process is creating the event or EPCIS data for receiving shown in 336 in FIG. 3A (or 376 in FIG. 3B). This data is in the industry standard format and may be later accessed for presentation in a dashboard for problem tracing. Example data is shown in FIG. 4.

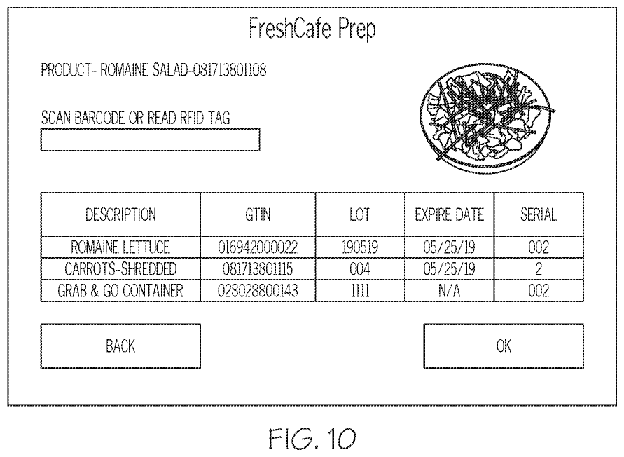

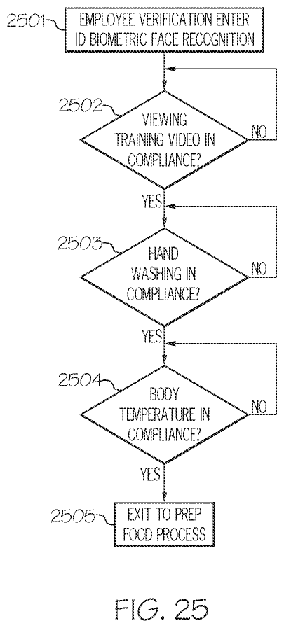

Referring to the sample input screen in FIG. 10. This input screen can be directly on a food prep printer capable of printing barcodes and encoding RFID or on a smart device that can communicate with a printer capable of printing barcodes and/or encoding RFID intelligent labels. Prior to preparation, the system will verify that the user is in compliance with employee guidance and best practices, such as shown the process map in FIG. 25. In the first step 2501, the user is prompted by the IoT printer to start the operation. Verification may be made in a number of ways, including, but not limited to, employee identification, biometric scan, facial recognition, and/or the like. The IoT printer will then review the configurable compliance checks, including whether or not: (1) the user is current on training compliance 2502; (2) the user is in compliance with required handwashing standard (verified by a smart device that can communicate directly with the IoT printer) 2503; and (3) the user has body temperature within a safe and healthy range 2504. The purpose of the check is two-fold: (1) to ensure user compliance; and (2) to provide an audit trail of compliance in case of a food quality incident (such as lack of compliance with 2502, 2503, and/or 2504) if the process exits in step 2505.

The user will select the item to prepare and then either barcode scan or RFID read all of the items required to prepare for the receipt. In addition, if the ability for RFID self-checkout is desired, the container can be scanned and be associated to the food product. In addition, if this is an intermediate step, the reusable container may be scanned. The scanning is an important step in the chain of custody by enabling a report to run showing all of the products that have been in the reusable container and any possible cross-contamination that may have occurred.

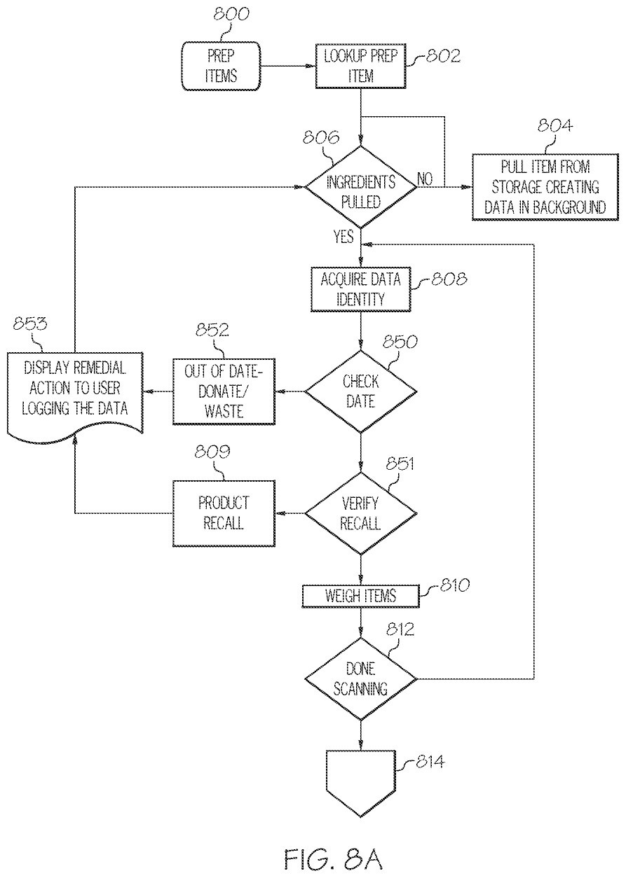

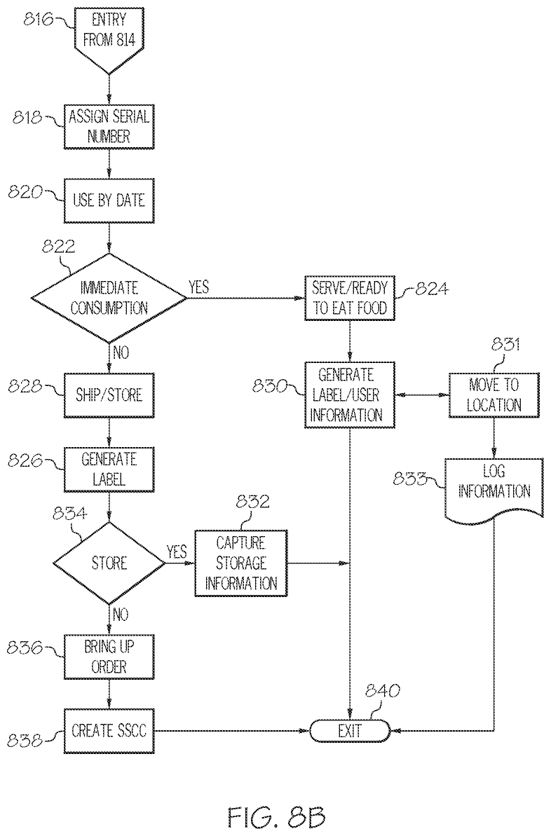

One potential embodiment of the transformation process is generally described in the flow chart depicted in FIGS. 8A and 8B and begins at 800 with a prepped food product item. At 802, the user looks up the food product item to be prepped. At 806, the user verifies that all of the food product item ingredients have been moved to the prep table, and at 808 the food items are scanned. At 850, the prep food product item ingredients are verified to ensure that sufficient time remains for it to be a viable ingredient. This verification is calculated by determining the remaining shelf life of the ingredient as compared to the desired shelf life of the prepped food product item. If there is not enough shelf life left to make the ingredient viable, then, at 852, the ingredient is processed for donation, re-purposing or properly disposed of as waste with the correct traceability information being generated, and the process returns to 806 to pull another inventory item.

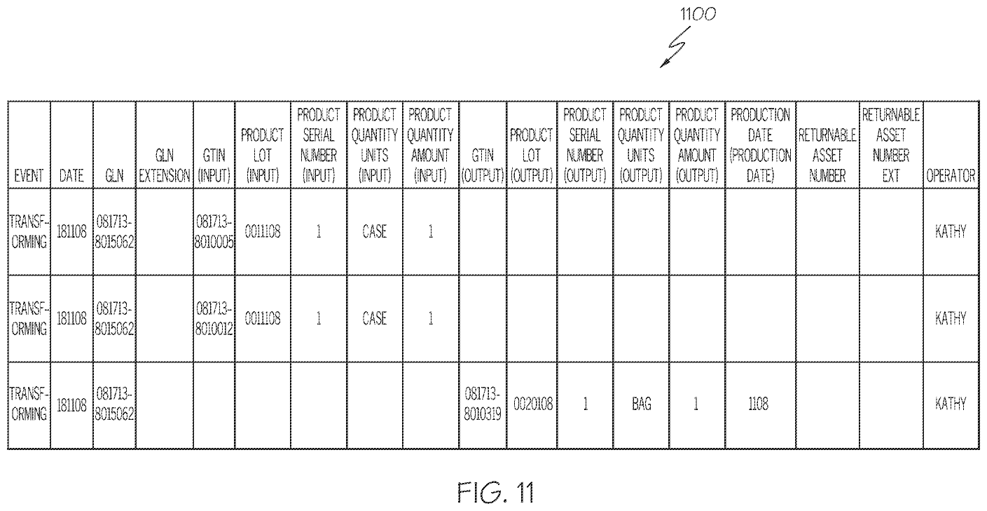

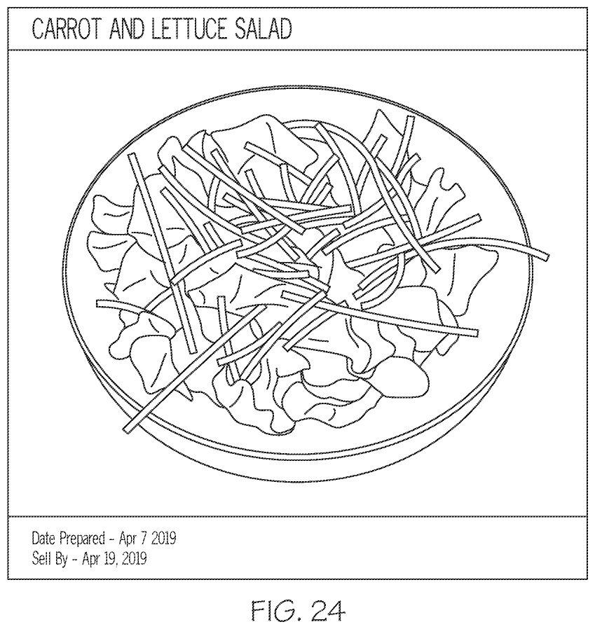

If the no path is taken to 809 the message or similar is displayed to the user, as shown in FIGS. 23 and 24 informing the user that remedial action needed because the food item is expired or recalled, respectively. In 853 the appropriate data is logged and the process returns to 806 to pull another inventory item. Following the yes path if all the items have been assembled each item is scanned and along with the option to weigh the item in 810. If the item will not be consumed in the prep process an accurate weight is required to maintain inventory. If all of the items have not been assembled, then the path to 814 is followed to assemble and then verify the items. In order to maintain the ease of use in the background the traceability information is being assembled as shown in FIG. 11. Thus far we have collected the unique identifier along with the batch/serial number and relevant data of the items inputted into the transformation process. This information is shown in the table in FIG. 11. Next we need to assign a serial number and a use by date to the newly created item. Following from 818 a serial number is generated. The serial number generated by the printer for the created item is appropriate for both a 2D barcode and a RAIN RFID 96 bit SGTIN by combining a serialized countered stored in the application with a 3-digit prefix for the device identifier. In 820 use by date is calculated for the prepped item by taking the earlier of the shelf life shown if FIG. 11 for the item or the ingredient with the first expiration date. In 822 the disposition of the prepped item is determined; if the item is to be served to a consumer the path to 824 is followed.

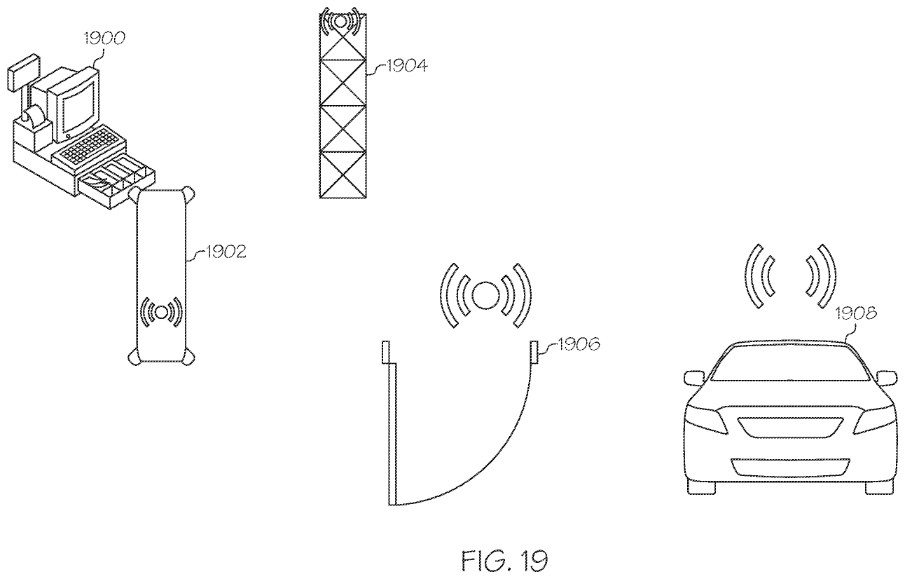

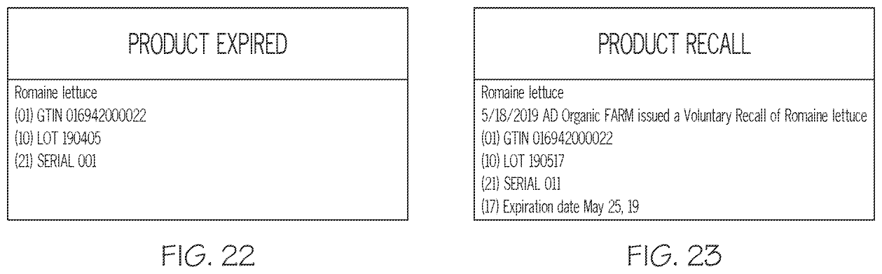

In FIG. 22 an example label prepared for an end consumer consumption is shown. Scanning the QR code on this label will link the consumer to a web page containing the information shown in FIG. 25. In 831 the move location is determined, which may be point-of-sale, grab & go or a delivery service to a customer. Each of these move locations has a different location assigned as shown in FIG. 19, which can be one of point-of-sale at the register 1900, an RFID reader 1902, grab-and-go 1904, an exit 1906, or a delivery service 1908. For both the grab-and-go area and the delivery service when the data is logged in the background the holding conditions are stored in the product log file. When the product is delivery or removed from holding for consumption a check of the recall status, expiration date and sensor data is verified. Then the progression is to ingredient put away shown in 840. Ingredient put away requires a weigh and scan out process. This process generates the traceability information moving the product back to the desired storage area. If there are empty containers, then the user designated the scanned item as disposed generating the appropriate traceability information. Then the process exits in 842.

Back in immediate consumption decision in 833 following the no path to 828 for ship/store process, the item level label is generated in 826. This the same label whether the item is stored or shipped. In the store decision 834, the yes path is followed to 832. The appropriate traceability information is generated and we progress to putting ingredients 840 away see above. If the no path is followed to 836 where the item will be immediately shipped, the purchase order is brought up on the printer and the item is scanned to the purchase order. This information will be used to generate and advance ship notice. If the order is complete in 838 serial shipping container code (SSCC) and an advance ship notice (ASN) are generated and progressed to 840 for putting ingredients or food items away.

Returning to step 850, if all of the necessary ingredients have been assembled and scanned and the dates verified at 850, then the user has the option of weighing the food product item at 810. If the food product item will not be consumed in the prep process, an accurate weight is required to maintain inventory. At 812, the item is done being scanned and the process exits to block 814 and enters the second half of the process depicted in FIG. 8B. If all of the items have not been assembled, then the path to 804 is followed to assemble and then verify the items/ingredients from storage. Additionally, in order to maintain the ease of use of the processes, the traceability information 1100 generated by the process depicted in FIG. 8A is concurrently being assembled in the background as depicted in FIG. 11 and includes the unique identifier along with the batch/serial number and relevant data of the items inputted into the transformation process. Next, the transformation process assigns a serial number and a “use by” date to the newly created item.

More specifically, as shown in FIG. 8B, the transformation process continues from block 814 to block 816. At 818, a serial number is generated and assigned to the food product item. The serial number is generated by the printer for the created item and is appropriate for, by way of example, both a 2D barcode and a RAIN RFID 96 bit SGTIN by combining a serialized counter stored in the application with a three-digit prefix for the device ID. Nonetheless, it is contemplated that other serial number generators can also be used to generate and assign serial numbers that satisfy user need and/or preference without affecting the overall concept of the present invention.

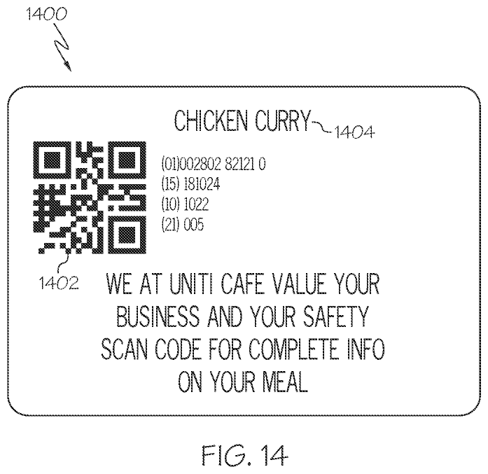

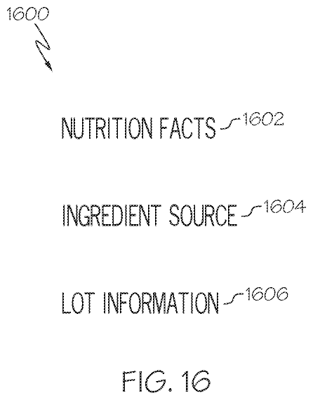

At 820, a “use by” date is calculated for the prepped food product item by using the earlier of the shelf life shown in FIG. 11 for the item, or the ingredient used in the food product item with the earliest expiration date. At 822, the disposition of the prepped food product item is determined. More specifically, if the food product item is to be served to a consumer for immediate consumption, the path to 824 is followed and, at 830, a label is generated with the appropriate traceability information. An example of a label 1400 prepared for immediate end consumer consumption is depicted in FIG. 14, wherein the label 1400 preferably comprises a QR code 1402 and an item description 1404. Scanning the QR code 1402 on label 1400 will link the user or consumer to a web page 1600 containing the information depicted in FIG. 16, such as nutrition facts 1602, ingredient source 1604, and/or lot information 1606. Of course, other useful information can also be linked and provided to the user to suit user need or preference.

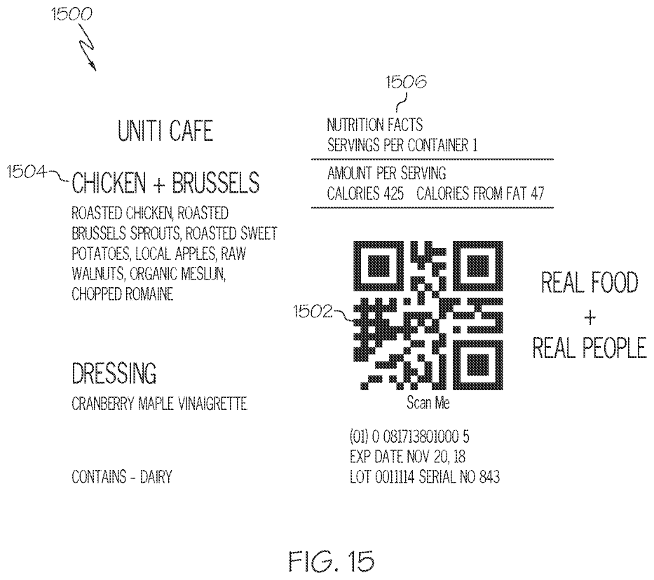

FIG. 15 also shows a front perspective view of one example of a quick serve label 1500 with traceability information that may be served with a food product item. The label 1500 preferably comprises a QR code 1502, an item description 1504, and nutrition information 1506. Scanning the QR code 1502 on label 1500 will also link the consumer to a web page 1600 containing the information shown in FIG. 16, or any additional information that may be desired, as disclosed above.

Returning now to FIG. 8B, after the label is generated, the process continues to 840 where the ingredients are put away. Specifically, putting the ingredient away preferably requires a weigh and scan out process. This process generates the traceability information moving the product back to the desired storage area. For example, if there are empty containers, then the user designates the scanned item as having been disposed of, and generates the appropriate traceability information. The process then exits at 842.

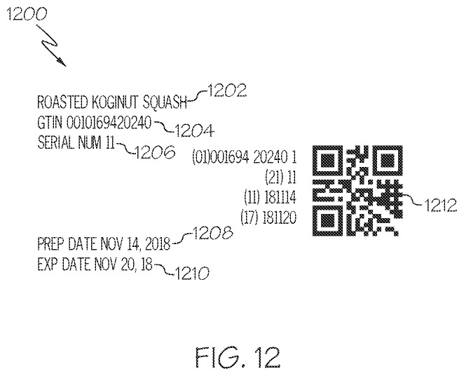

Returning now to the immediate consumption decision at 822 in FIG. 8B, if the food product item is not to be immediately served to a consumer, then the process continues to 828 the ship/store process, and the item level label is generated at 826. An example of this type of label is generally depicted at 1200 in FIG. 12. Label 1200 is the same whether the food product item is stored or shipped and typically comprises a food product item name 1202, a GTIN number 1204, a serial number 1206, a prep date 1208, an expiration date 1210, and a QR code 1212 that could link the user to a webpage that displays additional information about the food product item, its sourcing, traceability, etc.

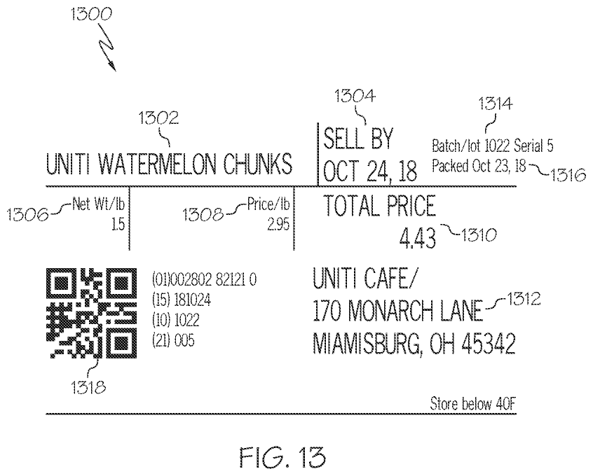

As the transformation process continues in FIG. 8B, it is determined if the food product item is to be stored or shipped at 834. If the item is stored, the process continues to 832. The appropriate traceability information is then generated and the process continues to 840 where the ingredients are put away as disclosed above, and the process exits at 842. If, on the other hand, the food product item is to be shipped and not stored, then the process continues to 836 where the purchase order is brought up on the printer and the food product item is scanned and linked to the purchase order. This information will then be used to generate and advance a ship notice label. FIG. 13 discloses one possible example of shipping label 1300, which preferably comprises an item name 1302, a “sell by” date 1304, a new wt./lb. 1306, a price/lb. 1308, a total price 1310, an address 1312, a batch/lot number 1314, a packed date 1316, and a QR code 1318 that could link the user to a webpage that displays additional information about the food product item, its sourcing, traceability, etc. If the order is complete at 838, a SSCC and an ASN are generated and the process continues to 840 where the ingredients are put away as disclosed above, and the process exists at 842. In a further embodiment of the method of the present invention, an alert can be generated to the party currently in possession of the food product item or ingredient when the expiration date is nearing or has passed, thereby reducing the likelihood that an expired food product item will be consumed.

For reference purposes, FIG. 22 shows representative intelligent labels. In this case, a salad with three ingredients or components—romaine lettuce, carrot and an empty intelligent container—to support enhance user experiences such as self-checkout. The output label that is shown is produced by an internet-of-things (IoT) printer.

Many other benefits will no doubt become apparent from future application and development of this technology.

All patents, applications, standards, and articles noted herein are hereby incorporated by reference in their entirety.

The present subject matter includes all operable combinations of features and aspects described herein. Thus, for example if one feature is described in association with an embodiment and another feature is described in association with another embodiment, it will be understood that the present subject matter includes embodiments having a combination of these features.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. As described hereinabove, the present subject matter solves many problems associated with previous strategies, methods/processes, systems and/or devices. However, it will be appreciated that various changes in the details, materials and arrangements of components, which have been herein described and illustrated in order to explain the nature of the present subject matter, may be made by those skilled in the art without departing from the principle and scope of the claimed subject matter, as expressed in the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A method of tracing a temperature profile of a food product from an origin to an ultimate disposition, comprising:
receiving at least one food item having a first set of data linked thereto;
wherein the first set of data comprises the temperature profile of the food item from the origin; and
wherein the first set of data is encoded in a machine-readable label;
comparing the temperature profile of the food item with an acceptable temperature range;
if the temperature profile is within the acceptable temperature range, transforming the at least one food item into a food product;
if the temperature profile is outside the acceptable temperature range, transforming the at least one food item into a food waste;
generating a second set of data linked to the food product or food waste;
wherein the second set of data is encoded in the machine-readable label; and
using the first and second sets of data to generate traceability information for the food product or the food waste.

2. The method of claim 1, further comprising:
providing the traceability information for the food product or food waste to a user.

3. The method of claim 1,
wherein the ultimate disposition of the food product is a consumer consumption or a re-purposing.

4. The method of claim 1,
wherein the ultimate disposition of the food waste is a disposal or a return to a supplier.

5. The method of claim 1,
wherein the first set of data further comprises a shelf life for each of the at least one food item and the second set of data further comprises an expiration date for the food product.

6. The method of claim 5,
wherein the shelf life for each of the at least one food item is used to calculate the expiration date for the food product.

7. The method of claim 1, further comprising:
using the temperature profile to generate an alert about the food product to a user.

8. The method of claim 1,
wherein the machine-readable label comprises at least one identifier selected from the group consisting a barcode, a RFID tag, and a QR code.

9. A method of identifying a recall for a food item between an origin to an ultimate disposition, comprising:
receiving at least one food item having a first set of data linked thereto;
wherein the first set of data comprises an identity of the food item; and
wherein the first set of data is encoded in a machine-readable label;
comparing the identity of the food item with a food recall list to generate a recall status;
if the identity of the food item is not on the food recall list, transforming the at least one food item into a food product;
if the identity of the food item is on the food recall list, transforming the at least one food item into a food waste;
generating a second set of data linked to the food product or food waste;
wherein the second set of data is encoded in the machine-readable label; and
using the first and second sets of data to generate traceability information for the food product or the food waste.

10. The method of claim 9, further comprising:
providing the recall status and traceability information for the food product or food waste to a user.

11. The method of claim 9,
wherein the ultimate disposition of the food product is a consumer consumption or a re-purposing.

12. The method of claim 9,
wherein the ultimate disposition of the food waste is a disposal or a return to a supplier.

13. The method of claim 9,
wherein the first set of data further comprises a shelf life for each of the at least one food item and the second set of data further comprises an expiration date for the food product.

14. The method of claim 13,
wherein the shelf life for each of the at least one food item is used to calculate the expiration date for the food product.

15. The method of claim 9, further comprising:
using the recall status to generate an alert about the food product to a user.

16. The method of claim 9,
wherein the machine-readable label comprises at least one identifier selected from the group consisting a barcode, a RFID tag, and a QR code.

17. A method for tracing a food item from a food service location to a consumer, comprising:
receiving the food item at the food service location;
assigning a set of critical information to the food item;
wherein the set of critical information comprises at least one of a temperature profile and a food recall status;
using the set of critical information to generate traceability information relative to the food item; and
encoding the traceability information in a machine-readable label.

18. The method of claim 17,
wherein the set of critical information further comprises one or more for the food item:
(a) a vendor identifier; (b) an identification number; (c) a description; (d) a storage location; (e) a shelf life; and (f) a weight.

19. The method of claim 17,
wherein the traceability information further comprises one or more of the following relative to the food product: (a) a list of ingredients; (b) a source of each of the list of ingredients;
(c) an identifying number; and (d) an expiration date.

20. The method of claim 17,
wherein the label comprises at least one identifier selected from the group consisting a barcode, a RFID tag, and a QR code.

21. The method of claim 17, further comprising:
assigning an initial destination to the food item and comprising the initial destination in the traceability information.

22. The method of claim 17, further comprising:
using the temperature profile, the recall status or a combination thereof to generate an alert about the food item for the consumer.

23. A method of ensuring employee compliance during preparation of a food product, comprising:
verifying the identity of an employee using an internet-of-thing printer;
evaluating compliance of the employee using the internet-of-thing printer;
wherein the compliance is selected from the criteria consisting of:
training compliance;
handwashing compliance;
body temperature within a safe and healthy range; and
combinations thereof;
wherein the handwashing compliance further comprising using a smart device; and
if the employee is compliant, permitting the employee to begin preparation of the food product.
